(19) United States
(12) Patent Application Publication
(10) Pub. No.: US 2012/0088978 A1
(43) Pub. Date: Apr. 12, 2012

(54) LAPAROSCOPE POUCH

(76) Inventors: Yung Su Park, Seoul (KR); Myoung Su Park, Gyeonggi-do (KR)

(21) Appl. No.: 12/900,062

(22) Filed: Oct. 7, 2010

(51) Int. Cl. A61B 1/32; A61B 17/22

(52) U.S. Cl. 600/204; 606/1

(57) ABSTRACT

A laparoscope pouch includes: a main shaft which is therein formed with a hollow; a handle shaft which is inserted and slidably moved in the main shaft; an arm which is disposed in one side of the handle shaft; a handle which is disposed in the other side of the handle shaft and is rotated relative to the handle shaft; an extraction bag which has an opening with its upper side opened, the arm inserted in the extraction bag to hold the opening; a lead which connects the extraction bag and the handle and fixes a position of the extraction bag by providing a tension to the extraction bag by rotation of the handle; and a slider which is coupled and fixed to the main shaft when the handle shaft is moved relative to the main shaft. The extraction bag can be tightly inserted and maintained in the arm by rotating the handle even during an operation, and the lead can be adjusted to an operation situation even if the lead is not properly arranged during manufacture.

### BACKGROUND OF THE INVENTION

1. Field of the Invention

The present invention relates to a laparoscope pouch, and more particularly, to a laparoscope pouch which is capable of adjusting a length of a lead connected to an extraction bag and a handle.

2. Description of the Related Art

In the related art, there have been pipes having disposable extraction bags used for laparoscope operation. Unfortunately, such pipes had to be imported from foreign countries and were so expensive that patent paid high expenses for even a simple laparoscopy operation. An imported organic extraction bag is configured in such a manner that, after an extraction pipe is led in through a trocar installed in a patent's abdomen for use, when a user pushes a grip of an inner portion of the extraction pipe with an outer portion thereof held in his/her hand, a circle-like spring is drawn out and the bag is opened to contain extracted things such as organs and the like.

### SUMMARY OF THE INVENTION

Accordingly, it is an object of the present invention to provide a laparoscope pouch which is capable of adjusting a length of a lead connected to an extraction bag and a handle.

It is another object of the present invention to provide a laparoscope pouch which is capable of preventing movement of a handle shaft during an operation through a handle lock interposed between a main shaft and the handle shaft.

To achieve the above objects, according to an aspect of the invention, there is provided a laparoscope pouch including: a main shaft which is therein formed with a hollow; a handle shaft which is inserted and slidably moved in the main shaft; an arm which is disposed in one side of the handle shaft; a handle which is disposed in the other side of the handle shaft and is rotated relative to the handle shaft; an extraction bag which has an opening with its upper side opened, the arm inserted in the extraction bag to hold the opening; a lead which connects the extraction bag and the handle and fixes a position of the extraction bag by providing a tension to the extraction bag by rotation of the handle; and a slider which is coupled and fixed to the main shaft when the handle shaft is moved relative to the main shaft.

Preferably, a cylindrical rotating part for rotation relative to the handle is formed in the other side of the handle shaft and a rotation groove conforming to the rotating part is formed in the handle.

Preferably, a handle lock to make an interlock with the handle shaft is further interposed between the main shaft and the handle shaft.

Preferably, the handle lock includes: a locking member which is disposed on the main shaft and is actuated by a handling power from a user to selectively interlock with the handle shaft; an interfering part which is disposed on the handle shaft and makes an interlock with the locking member; and an elastic member which is interposed between the locking member and the main shaft and provides an elastic force to the locking member.

Preferably, at least one of the interfering part and the locking member is formed to have a shape of one-way clutch to generate a locking in one direction only.

Preferably, the locking member includes: a handling part which is exposed through the main shaft and to which the handling power from the user is applied; a locking part which makes an interlock with the handle shaft; and a rotating part which is interposed between the handling part and the locking part and forms a rotation center such that the rotating part is rotated by the handling power.

Preferably, the handle shaft includes: a lead groove in which the lead is inserted; and at least one hole through which the lead passes.

Preferably, the handle includes: a rotation groove in which the other side of the handle shaft is inserted and which is rotated relative to the handle shaft; and a lead fixing part to which the lead is fixed.

Preferably, the arm is disposed to pass through the slider.

According to another aspect of the invention, there is provided a laparoscope pouch including: a main shaft which is therein formed with a hollow; a handle shaft which is inserted and slidably moved in the main shaft; an arm which is disposed in one side of the handle shaft; a handle which is disposed in the other side of the handle shaft and is rotated relative to the handle shaft; an extraction bag which has an opening with its upper side opened, the arm inserted in the extraction bag to hold the opening; a lead which connects the extraction bag and the handle and fixes a position of the extraction bag by providing a tension to the extraction bag by rotation of the handle; and a handle lock which is interposed between the main shaft and the handle shaft to make an interlock with the handle shaft, wherein the handle lock includes: a locking member which is disposed on the main shaft and is actuated by a handling power from a user to selectively interlock with the handle shaft; an interfering part which is disposed on the handle shaft and makes an interlock with the locking member; and an elastic member which is interposed between the locking member and the main shaft and provides an elastic force to the locking member, and wherein the handle includes: a rotation groove in which the other side of the handle shaft is inserted and which is rotated relative to the handle shaft; and a lead fixing part to which the lead is fixed.

Preferably, a cylindrical rotating part for rotation relative to the handle is formed in the other side of the handle shaft and a rotation groove conforming to the rotating part is formed in the handle.

Preferably, at least one of the interfering part and the locking member is formed to have a shape of one-way clutch to generate a locking in one direction only.

Preferably, the locking member includes: a handling part which is exposed through the main shaft and to which the handling power from the user is applied; a locking part which makes an interlock with the handle shaft; and a rotating part which is interposed between the handling part and the locking part and forms a rotation center such that the rotating part is rotated by the handling power.

The laparoscope pouch according to the present invention has an advantage that, since a length of the lead connected to the extraction bag is adjusted by rotating the handle to which the lead is connected, the extraction bag can be tightly inserted and maintained in the arm by rotating the handle even during an operation, and a user can adjust the lead to an operation situation even if the lead is not properly arranged during manufacture.

In addition, the laparoscope pouch according to the present invention has another advantage that, since the handle shaft to which the arm is connected through the handle lock is fixed, the handle shaft is integrated with the main shaft during an operation so that the handle shaft cannot be slid, and thus the extraction bag can be prevented from being wrongly separated from the arm during the operation.

### BRIEF DESCRIPTION OF THE DRAWINGS

The above and/or other aspects and advantages of the present invention will become apparent and more readily appreciated from the following description of the embodiments, taken in conjunction with the accompanying drawings of which:

FIG. 1 is a sectional front view showing a laparoscope pouch according to one embodiment of the present invention;

FIG. 2 is a sectional side view of the laparoscope pouch shown in FIG. 1;

FIG. 3 is a sectional front view of a handle shaft shown in FIG. 1;

FIG. 4 is a sectional side view of FIG. 2;

FIG. 5 is a front view of a handle shown in FIG. 1;

FIG. 6 is a front view of a locking member shown in FIG. 2;

FIG. 7 is a plan view of the locking member shown in FIG. 6;

FIG. 8 is a plan view of a main grip shown in FIG. 1;

FIG. 9 is an enlarged view of portion “A” of FIG. 1;

FIG. 10 is an enlarged view of portion “B” of FIG. 1;

FIG. 11 is an enlarged view of portion “C” of FIG. 2;

FIG. 12 is an enlarged view of portion “D” of FIG. 2; and

FIG. 13 is a sectional view showing a locking mechanism according to another embodiment of the present invention.

### DETAILED DESCRIPTION OF THE PREFERRED EMBODIMENT

Hereinafter, preferred embodiments of the present invention will be described with reference to the accompanying drawings.

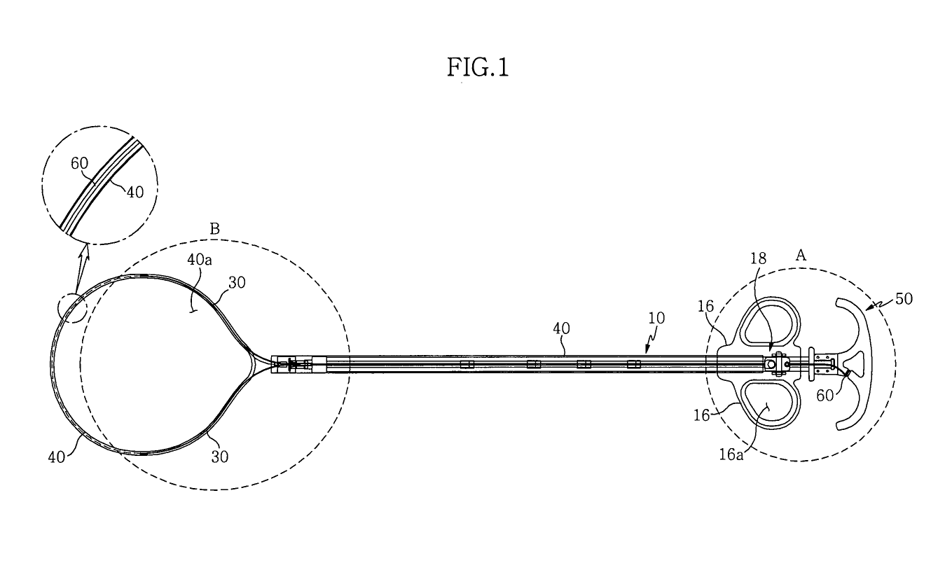

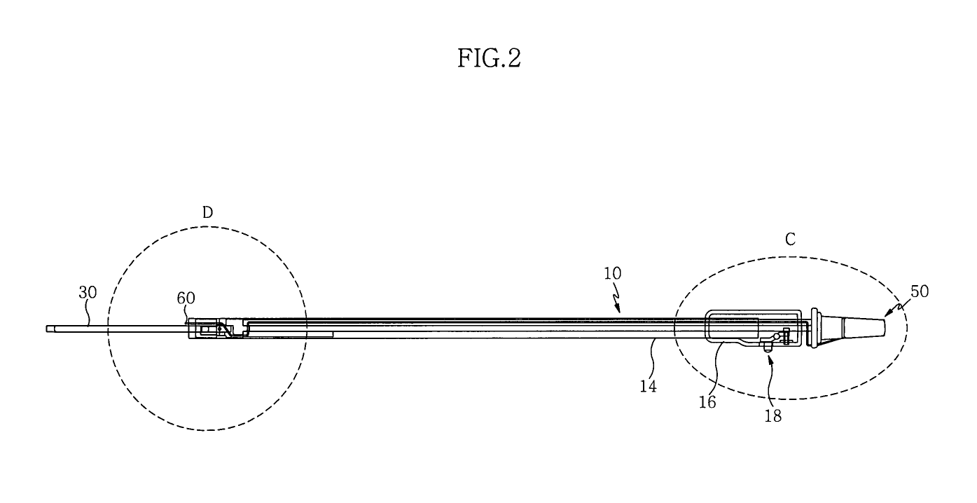

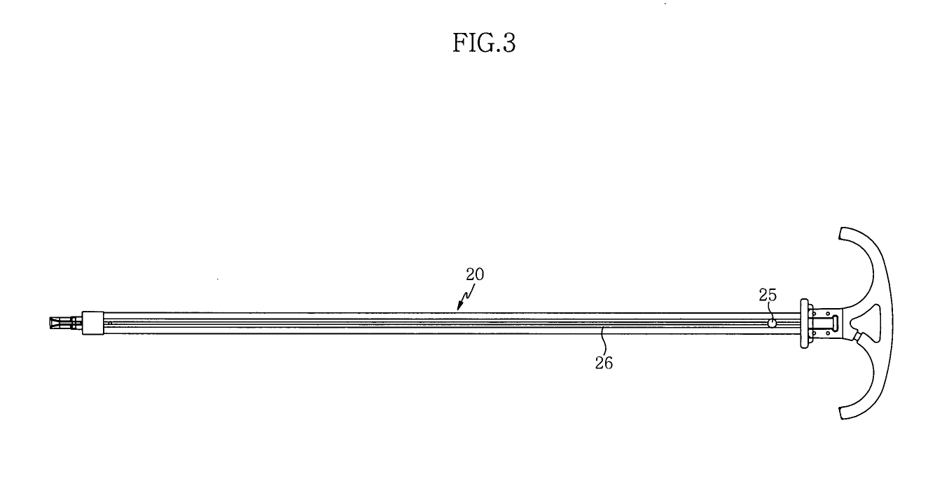

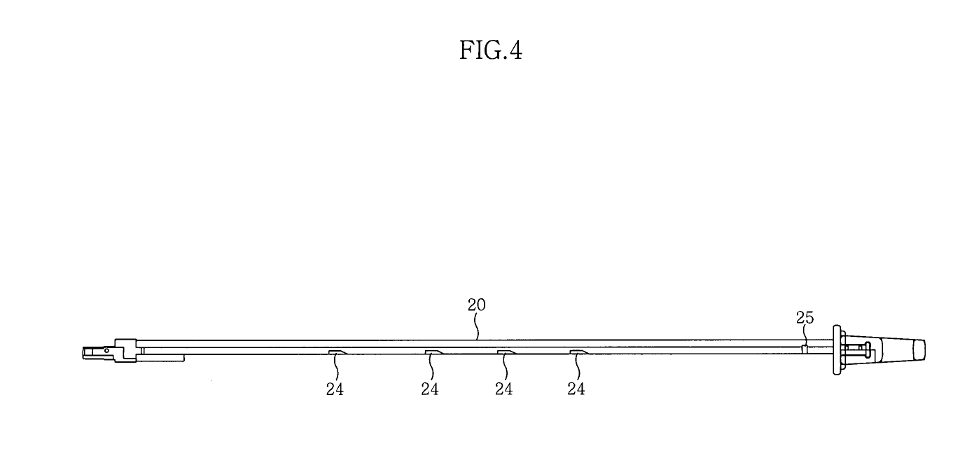

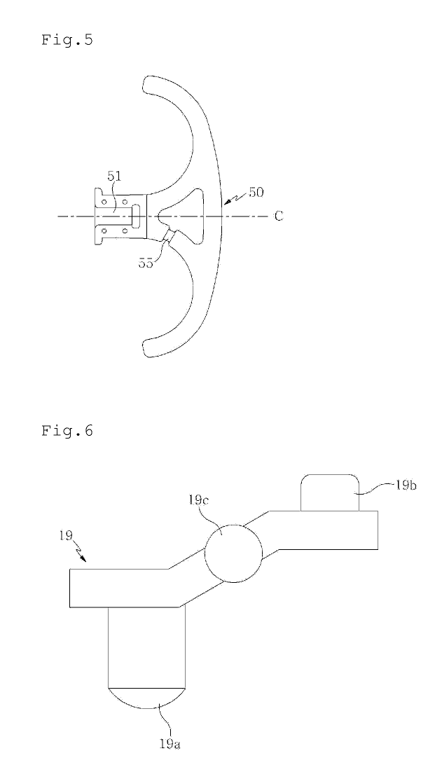

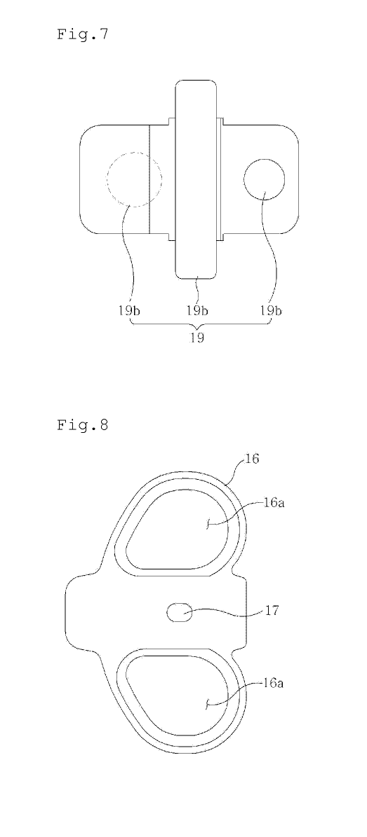

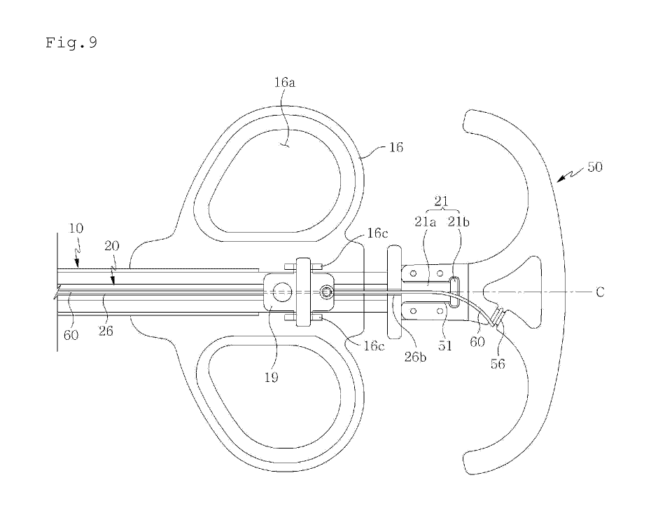

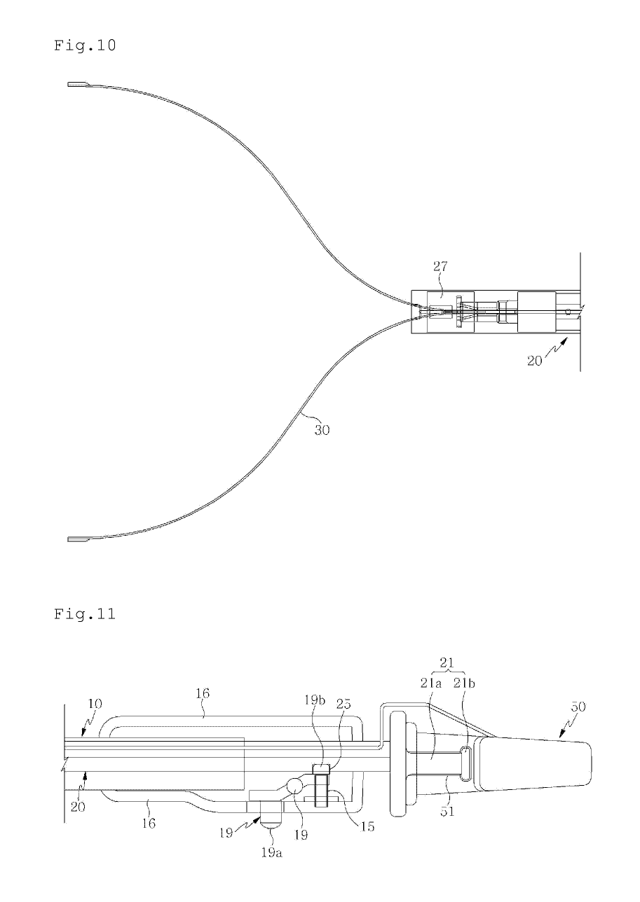

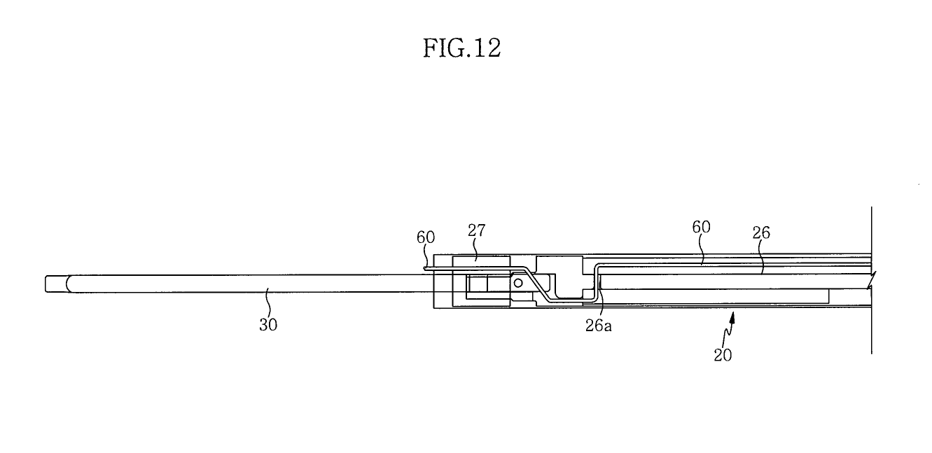

FIG. 1 is a sectional front view showing a laparoscope pouch according to one embodiment of the present invention, FIG. 2 is a sectional side view of the laparoscope pouch shown in FIG. 1, FIG. 3 is a sectional front view of a handle shaft shown in FIG. 1, FIG. 4 is a sectional side view of FIG. 2, FIG. 5 is a front view of a handle shown in FIG. 1, FIG. 6 is a front view of a locking member shown in FIG. 2, FIG. 7 is a plan view of the locking member shown in FIG. 6, FIG. 8 is a plan view of a main grip shown in FIG. 1, FIG. 9 is an enlarged view of portion “A” of FIG. 1, FIG. 10 is an enlarged view of portion “B” of FIG. 1, FIG. 11 is an enlarged view of portion “C” of FIG. 2, and FIG. 12 is an enlarged view of portion “D” of FIG. 2.

Referring to FIGS. 1 to 12, a laparoscope pouch 100 according to an embodiment of the present invention includes a main shaft 10 having a hollow 12 formed therein, a handle shaft 20 which is inserted inside the main shaft 10 and is slidably moved, an arm 30 which is disposed on one side of the handle shaft 20, a handle 50 which is disposed on the other side of the handle shaft 20 and is rotated relative to the handle shaft 20, an extraction bag 40 in which the arm 30 is inserted, and a lead 60 which connects the extraction bag 40 and the handle 50 and has a length adjusted depending on the number of rotations of the handle 50.

The main shaft 10 includes a pipe 14 in which the handle shaft 20 is inserted and slidably moved, a main grip which is disposed on one side of the pipe 14, and a handle lock 18 which is disposed on the main grip 16 and selectively limits the slidable movement of the handle shaft 20.

The pipe 14 has therein the hollow 12 through which the handle shaft 20 is moved relative to the pipe 14.

In this embodiment, a section perpendicular to an axial direction of the handle shaft 20 is not cylindrical but atypical such that the handle shaft 20 is not rotated when the handle 50 is rotated, and the hollow 12 of the main shaft 10 is configured to have a section conforming to the handle shaft 20.

The main grip 16 serves to allow a user to hold and fix the main shaft 10 for an operation. The main grip 16 has a grip hole 16a in which fingers can be inserted, and a penetrating handle lock hole 17. S portion of the handle lock 18 is exposed to the outside of the main grip 16 through the handle lock hole 17 so that the user can apply a handling power.

The handle lock 18 is disposed on the main grip 16 and includes a locking member 19 which is actuated by the handling power from the user to make an interlock with the handle shaft 20, and an elastic member 15 which is interposed between the locking member 19 and the main grip 16 and provides an elastic force to the locking member 19.

The locking member 19 includes a handling part 19a which projects through the handle lock 17, a locking part 17b which makes an interlock with the handle shaft 20, and a rotating part 19c which is disposed on the main grip 16 to be rotated by the handling power from the user.

The handling part 19a and the locking part 19b are formed to be in the opposition to each other. In this embodiment, the elastic member 15 is interposed between the locking part 19b and the main grip 16. In addition, a supporting part 16c in which the rotating part 19c is inserted is formed inside the main grip 16. In this embodiment, the elastic member 15 is implemented by a coil spring. Thus, when the user applies a handling power to the handling part 19a, the locking member 19 is rotated around the rotating part 19c while compressing the elastic member 15, thereby releasing an interlock between the locking part 19b and an interfering part 25 of the handle shaft 20 and freely moving the handle shaft 20 relative to the main shaft 10.

Specifically, the main grip 16 is therein formed with an inner hollow (not shown) through which the handle shaft 20 can be inserted and relatively moved. The hollow of the main grip 16 is configured to communicate with the hollow 12 of the main shaft 10.

Next, the handle shaft 20 has a shape of elongated bar and is therein formed with interfering parts 24 and 25 which make an interlock with the handle lock 19. In this embodiment, the interfering parts 24 and 25 may have a groove shape or any other shape. In addition, the interfering part 24 limits a length of drawing-out of the handle shaft 20 by making an interlock with the locking part 19b when the handle shaft 20 is drawn out.

In addition, the handle shaft 20 is formed therein with a lead groove in which a lead 60 connected to the extraction bag is inserted. One side of the lead 60 is fixed to the extraction bag 40 through a first hole 26a formed at a side of extraction bag 40 formed on the handle shaft 20 and the other side of the lead 60 is fixed to the handle 50 through a second hole 26b formed in the handle 50.

The arm 30 is inserted and disposed in one side end of the handle shaft 20 and the handle 50 is disposed in the other side end thereof so that the handle 50 can be rotated relative to the handle shaft 20.

In this embodiment, the arm 30 is made of a shape memory alloy and is formed to open into a shape of tongs so that the extraction bag 40 keeps in its own shape. The arm is located inside the main shaft 10 before use and projects toward one side of the main shaft 10 by user's handling for use only.

In addition, a cylindrical rotating part 21 is formed on the other side end of the handle shaft 20 so that the handle 50 can be relatively rotated. The rotating part 21 includes a cylindrical portion 21a which is formed to extending toward a side of handle 50 and a locking portion 21b which prevents the cylindrical portion 21a from separating from the handle 50 in an axial direction. The locking portion 21b is formed to be larger in radius than the cylindrical portion 21a. In this embodiment, the handle includes a rotation groove 51 formed to conform to the rotation part 21. Thus, the rotating part 21 is rotated around an axis with it inserted in the rotation groove 51, but is not separated from the handle 50 in a sliding movement direction of the handle shaft 20.

In addition, on the one side end of the handle shaft 20 is disposed a slider 27 for coupling to the main shaft 10 at initial movement of the slider 27 to the one side end of the handle shaft 20. The slider 27 is inserted through the other side of the main shaft 10 and is moved to the one side end of the handle shaft 20 and is configured to couple to a coupling groove (not shown) formed on the one side end of the main shaft 10. After coupling to the coupling groove, the slider 27 is fixed to the main shaft 10 so as not to generate the relative movement.

In this embodiment, the arm 30 and the lead 60 are disposed to pass through the slider 27 and are configured to relatively move, with only the slider 27 fixed, when the handle shaft 20 is slidably moved.

In addition, a rotating part groove 51 corresponding to the rotating part 21 is formed in the handle 50.

In addition, the handle 50 has a lead fixing part 56 to which the lead 60 is fixed. The lead fixing part 56 is located outside the rotation center C of the handle 50 and is disposed to allow the lead 60 to be easily wound when the handle 50 is rotated.

In the meantime, the extraction bag 40 is formed to have a shape of bag with its upper side opened. The lead 60 is disposed with a shape of snare on the upper side of the extraction bag 40. When the lead 60 is pulled, a tension is provided to pull an opening 40a of the extraction bag 40.

In addition, the arm 60 is configured to be separated from the extraction bag 40 by user's handling when the arm 60 is inserted in the extraction bag 40.

Hereinafter, operation of the laparoscope pouch will be described in more detail with reference to FIGS. 1, 2 and 7 to 10.

First, before a user actuates the laparoscope pouch 100, the handle shaft 20 is located inside the main shaft 10.

Here, the extraction bag 40 is located inside the main shaft 10. This is to prevent organs or bloods contained in the extraction bag 40 from being leaked out by preventing the extraction bag 40 inserted in a body of a patient from being infected or damaged.

Next, for use of the laparoscope pouch 100, when a user pushes and moves the handle 50 toward the main grip 16, the slider 27 disposed on the one side end of the handle shaft 20 is slidably moved within the main shaft 10 and fixed to the main shaft 10, and the extraction bag 40 disposed on the one side end of the handle shaft 20 projects and opens toward one side of the main shaft 10 (FIG. 1).

Then, the arm 30 inserted in the extraction bag 40 is opened by an elastic force such that the opening 40a of the extraction bag 40 is expanded.

In particular, the relative movement in the handle shaft 20 is limited by generating mutual interference between the locking member 19 disposed on the main shaft 10 and the interfering part 25 after the slidable movement of the handle shaft 20.

In other words, the movement of the handle shaft 20 is limited by the locking member 19 and thus the movement of the arm 30 fixed to the handle shaft 20 is limited, thereby preventing the extension bag 40 and the arm 30 from being separated from each other due to any wrong handling of the user.

In addition, as the arm 30 is opened when the handle shaft 20 is slidably moved, an operation can be performed with an incision of a human body minimized, which may result in fast recovery of a patient.

In the meantime, the handle 50 to which the lead 60 is fixed can fix a position of the extraction bag 40 in which the arm 30 is inserted, which may result in prevent of the extraction bag 40 from being separated from the arm 30.

Here, if the lead 60 interposed between the extraction bag 40 and the handle 50 is too long, the extraction bag 40 may be separated from the arm 30 due to weight of organs contained in the extraction bag 40. In the past, a user had to fix the lead connected to the extraction bag and the handle shaft and then check length of the lead in detail. However, the laparoscope pouch 100 according to this embodiment is configured to allow the user to adjust the length of the lead 60 even if it is a bit too lengthy.

Thus, when the user rotates the handle 50, the fixing part 56 to which the lead 60 is fixed is rotated to allow the lead 60 to be wound on the periphery of the handle 50. If the lead 60 is excessively wound, the opening 40a of the extraction bag 40 is closed to make its area narrower, and in case of which, the opening area of the extraction bag 40 can be recovered to its original condition by rotating the handle 50 in the direction in the opposite to the wound direction.

Thereafter, organs separated from a human body in a laparoscopy operation are contained in the extraction bag 40.

Next, a process of removing the laparoscope pouch according to this embodiment will be described.

First, the extraction bag 40 is located inside a human body and the handle 50 is located outside the human body. In this case, since a volume of the extraction bag 40 is larger than that of an incision, the extraction bag 40 cannot be drawn out with it opened.

Therefore, a user fixes the main shaft 10, pushes the locking member 19 so that the hand shaft 20 can be slidably moved, and pulls the handle 50.

In this case, the lead 60 and the arm 30 are pulled through the one end of the main shaft 10, the extraction bag 40 closes the opening 40a opened by the pulled lead 60 in a shape of snare, and the arm 30 is separated from the extraction bag 40 and is moved inside the main shaft 10.

Here, the locking part 19b is located in the fixing part 56 to prevent the handle shaft 20 from being drawn out at a time.

Thereafter, the user cuts the lead 60 to be separated from the handle 50 and separates the separated handle 50 and the handle shaft 20 from the main shaft 10, leaving only the main shaft 10 and the extraction bag 40.

Then, the user leaves only the extraction bag 40 connected to the lead 60 by removing the main shaft 20 from a human body through the main grip 16.

Thereafter, the user completes removal of the laparoscope pouch 100 by pulling the lead 60 to draw the extraction bag 40 out of the human body.

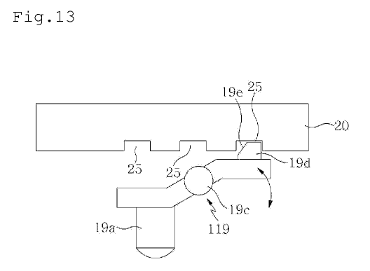

FIG. 13 is a sectional view showing a locking mechanism according to another embodiment of the present invention.

As shown in FIG. 13, a locking mechanism 119 according to this embodiment is configured to have a shape of one-way clutch to generate a locking only in a drawing-out direction, not in an entrance direction when the handle shaft 20 is slidably moved.

Specifically, by forming a locking part 19d formed with one-way clutching means such as an inclined plane 19e or a round (not shown) instead of the locking part 19b of the earlier embodiment, the locking mechanism 119 is configured such that interlock between the handle shaft 20 and the locking part 19d is generated only when the handle shaft 20 without it being generated due to the inclined plane 19e when the handle shaft 20 is inserted.

This embodiment has the same remaining configurations as the earlier embodiment, and therefore detailed explanation of which is not repeated.

While the present invention has been particularly shown and described with reference to exemplary embodiments thereof, it will be understood by those skilled in the art that various changes in form and details may be made therein without departing from the spirit and scope of the present invention. The exemplary embodiments are provided for the purpose of illustrating the invention, not in a limitative sense. Thus, it is intended that the present invention covers the modifications and variations of this invention provided they come within the scope of the appended claims and their equivalents.

What is claimed is:

1. A laparoscope pouch comprising:
a main shaft which is therein formed with a hollow;
a handle shaft which is inserted and slidably moved in the main shaft;
an arm which is disposed in one side of the handle shaft;
a handle which is disposed in the other side of the handle shaft and is rotated relative to the handle shaft;
an extraction bag which has an opening with its upper side opened, the arm inserted in the extraction bag to hold the opening;
a lead which connects the extraction bag and the handle and fixes a position of the extraction bag by providing a tension to the extraction bag by rotation of the handle; and
a slider which is coupled and fixed to the main shaft when the handle shaft is moved relative to the main shaft.

2. The laparoscope pouch according to claim 1, wherein a cylindrical rotating part for rotation relative to the handle is formed in the other side of the handle shaft and a rotation groove conforming to the rotating part is formed in the handle.

3. The laparoscope pouch according to claim 1, wherein a handle lock to make an interlock with the handle shaft is further interposed between the main shaft and the handle shaft.

4. The laparoscope pouch according to claim 3, wherein the handle lock includes:
a locking member which is disposed on the main shaft and is actuated by a handling power from a user to selectively interlock with the handle shaft;
an interfering part which is disposed on the handle shaft and makes an interlock with the locking member; and
an elastic member which is interposed between the locking member and the main shaft and provides an elastic force to the locking member.

5. The laparoscope pouch according to claim 4, wherein at least one of the interfering part and the locking member is formed to have a shape of one-way clutch to generate a locking in one direction only.

6. The laparoscope pouch according to claim 4, wherein the locking member includes:
a handling part which is exposed through the main shaft and to which the handling power from the user is applied;
a locking part which makes an interlock with the handle shaft; and
a rotating part which is interposed between the handling part and the locking part and forms a rotation center such that the rotating part is rotated by the handling power.

7. The laparoscope pouch according to claim 1, wherein the handle shaft includes:
a lead groove in which the lead is inserted; and
at least one hole through which the lead passes.

8. The laparoscope pouch according to claim 1, wherein the handle includes:
a rotation groove in which the other side of the handle shaft is inserted and which is rotated relative to the handle shaft; and
a lead fixing part to which the lead is fixed.

9. The laparoscope pouch according to claim 1, wherein the arm is disposed to pass through the slider.

10. A laparoscope pouch comprising:
a main shaft which is therein formed with a hollow;
a handle shaft which is inserted and slidably moved in the main shaft;
an arm which is disposed in one side of the handle shaft;
a handle which is disposed in the other side of the handle shaft and is rotated relative to the handle shaft;
an extraction bag which has an opening with its upper side opened, the arm inserted in the extraction bag to hold the opening;
a lead which connects the extraction bag and the handle and fixes a position of the extraction bag by providing a tension to the extraction bag by rotation of the handle; and
a handle lock which is interposed between the main shaft and the handle shaft to make an interlock with the handle shaft,
wherein the handle lock includes:
a locking member which is disposed on the main shaft and is actuated by a handling power from a user to selectively interlock with the handle shaft;
an interfering part which is disposed on the handle shaft and makes an interlock with the locking member; and
an elastic member which is interposed between the locking member and the main shaft and provides an elastic force to the locking member, and
wherein the handle includes:
a rotation groove in which the other side of the handle shaft is inserted and which is rotated relative to the handle shaft; and
a lead fixing part to which the lead is fixed.

11. The laparoscope pouch according to claim 10, wherein a cylindrical rotating part for rotation relative to the handle is formed in the other side of the handle shaft and a rotation groove conforming to the rotating part is formed in the handle.

12. The laparoscope pouch according to claim 10, wherein at least one of the interfering part and the locking member is formed to have a shape of one-way clutch to generate a locking in one direction only.

13. The laparoscope pouch according to claim 10, wherein the locking member includes:
a handling part which is exposed through the main shaft and to which the handling power from the user is applied;
a locking part which makes an interlock with the handle shaft; and
a rotating part which is interposed between the handling part and the locking part and forms a rotation center such that the rotating part is rotated by the handling power.
